(19) United States
(12) Patent Application Publication
(10) Pub. No.: US 2026/0108230 A1
(43) Pub. Date: Apr. 23, 2026

(54) ULTRASOUND TRANSDUCER AND METHOD OF WAFER SCALE FABRICATION OF TRANSDUCERS

(71) Applicant: GE Precision Healthcare LLC, Waukesha, WI (US)

(72) Inventors: Alexis HUBERT, Antibes (FR); Carole GADOIS, Antibes (FR); Sihem LAZIZI, Mougins (FR); Fereshteh ORDOKHANI, Antibes (FR)

(21) Appl. No.: 18/923,882

(22) Filed: Oct. 23, 2024

(51) Int. Cl. A61B 8/00; G01S 7/52

(52) U.S. Cl. CPC A61B 8/4494; G01S 7/52019; G01S 7/52023; A61B 2562/12

(57) ABSTRACT

A transducer of an ultrasound probe includes a piezoelectric layer configured to transmit an ultrasound signal towards a region of interest of a subject to be imaged, and receive an echo signal reflected by the region of interest of the subject to be imaged. The transducer includes a first electrode provided on a first surface of the piezoelectric layer, and a second electrode provided on a second surface of the piezoelectric layer. The transducer includes a circuit that is electrically connected to the first electrode and the second electrode. The transducer includes a frame that is provided on at least a third surface of the piezoelectric layer, and that electrically connects the first electrode to the circuit.

### TECHNICAL FIELD

The present disclosure relates to an ultrasound transducer including a piezoelectric layer, a first electrode provided on the piezoelectric layer, a second electrode provided on the piezoelectric layer, a circuit that is electrically connected to the first electrode and the second electrode, and a frame that electrically connects the first electrode to the circuit. Further, the present disclosure relates to a method of wafer scale fabrication of ultrasound transducers using a frame array.

### BACKGROUND

A piezoelectric layer may be provided in a transducer of an ultrasound probe for various ultrasound applications, such as ultrasound imaging, ultrasound measurements, tissue ablation, ultrasound diagnosis, or the like. The piezoelectric layer may be formed of a piezoelectric material that deforms in response to an electrical signal in order to generate an ultrasound signal, and/or that deforms in response to an echo signal in order to generate an electrical signal. Single-crystal piezoelectric materials (e.g., PMN-PT, PIN-PMN-PT, etc.) may be used for the piezoelectric layer because of relatively greater performance (e.g., greater piezoelectric charge coefficient, greater electromechanical coupling factor, etc.) as compared to PZT materials. The crystal growing process (e.g., melt method, Bridgman method, etc.) of single-crystal piezoelectric materials results in a relatively small boule or ingot, which limits the dimensions of the single-crystal piezoelectric material. Accordingly, the fabrication of a large number of transducers from these single-crystal piezoelectric materials may be inefficient, time-consuming, expensive, difficult, etc., due to the dimensions of the single-crystal piezoelectric materials.

Some transducers of ultrasound probes include “wrap-around” electrodes. In this configuration, a piezoelectric layer includes a signal electrode provided on a top surface of the piezoelectric layer, and a ground electrode provided on a bottom surface of the piezoelectric layer, or vice versa. In some cases, the side surfaces of the piezoelectric layer may be metallized using sputtering, electro-deposition, plating, controlled dispensing, jetting, etc., to electrically connect the signal electrode to a circuit (e.g., a flexible circuit, a printed circuit board (PCB), etc.). However, the metallized side surfaces are prone to flaking, chipping, or the like. In other cases, the side surfaces of the piezoelectric layer may be layered with a silver-epoxy to electrically connect the signal electrode to the circuit. However, silver-epoxy has low chemical resistance and can degrade over time. In these cases, the degradation of the side surfaces can induce signal issues, noise, etc., and negatively affect the reliability and performance of the transducer.

### SUMMARY

This summary introduces concepts that are described in more detail in the detailed description. It should not be used to identify essential features of the claimed subject matter, nor to limit the scope of the claimed subject matter.

In an aspect, a transducer of an ultrasound probe may include a piezoelectric layer configured to transmit an ultrasound signal towards a region of interest of a subject to be imaged, and receive an echo signal reflected by the region of interest of the subject to be imaged; a first electrode provided on a first surface of the piezoelectric layer; a second electrode provided on a second surface of the piezoelectric layer; a circuit that is electrically connected to the first electrode and the second electrode; and a frame that is provided on at least a third surface of the piezoelectric layer, and that electrically connects the first electrode to the circuit.

According to an embodiment, the frame may be provided on the third surface of the piezoelectric layer and a fourth surface of the piezoelectric layer.

According to an embodiment, the frame may be provided on the third surface of the piezoelectric layer, a fourth surface of the piezoelectric layer, a fifth surface of the piezoelectric layer, and a sixth surface of the piezoelectric layer.

According to an embodiment, the transducer is a one-dimensional array of transducer elements.

According to an embodiment, the transducer may include one or more acoustic matching layers configured to reduce an acoustic impedance between the subject and the transducer.

According to an embodiment, the transducer may include an acoustic dematching layer configured to attenuate the ultrasound signal, wherein the second electrode is provided between the acoustic dematching layer and the piezoelectric layer.

According to an embodiment, the transducer may include a lens configured to direct the ultrasound signal towards the region of interest of the subject.

According to an embodiment, the transducer may include a backing layer configured to attenuate ultrasound signals directed from the piezoelectric layer in a direction opposite to the subject.

According to an embodiment, the transducer may be formed by: providing a frame array, including the frame, on a carrier, providing the piezoelectric layer in a slot of the frame array, and singulating the transducer, including the frame, from the frame array.

According to an embodiment, the carrier may be an acoustic matching layer, an acoustic dematching layer, or a sacrificial substrate that is not a part of the transducer.

According to an embodiment, the transducer may be formed by: providing a frame array including a plurality of slots on a carrier, providing a plurality of piezoelectric layers in respective slots of the plurality of slots to form an array of transducers, and singulating the transducer from the array of transducers.

According to an embodiment, the piezoelectric layer may be formed of a single crystal piezoelectric material.

According to an embodiment, sidewalls of the piezoelectric layer might not be metallized.

According to an embodiment, the frame may be a portion of a frame array used during manufacturing of the transducer, the frame array may include a plurality of slots corresponding to respective transducers, and the piezoelectric layer may be provided in a slot, of the plurality of slots, of the frame array.

According to an embodiment, the frame may be formed of an electrically conductive material.

According to an embodiment, the frame may be formed of graphite.

In another aspect, an ultrasound probe may include a transducer that includes a piezoelectric layer configured to transmit an ultrasound signal towards a region of interest of a subject to be imaged, and receive an echo signal reflected by the region of interest of the subject to be imaged; a first electrode provided on a first surface of the piezoelectric layer; a second electrode provided on a second surface of the piezoelectric layer; a circuit that is electrically connected to the first electrode and the second electrode; and a frame that is provided on at least a third surface of the piezoelectric layer, and that electrically connects the first electrode to the circuit.

In yet another aspect, a method of manufacturing a transducer of an ultrasound probe may include providing a frame array on a carrier, wherein the frame array includes a plurality of slots; providing a plurality of piezoelectric layers in respective slots of the plurality of slots to form an array of transducers; and singulating the transducer from the array of transducers, wherein each transducer of the array of transducers comprises: a respective piezoelectric layer, of the plurality of piezoelectric layers, configured to transmit an ultrasound signal towards a region of interest of a subject to be imaged, and receive an echo signal reflected by the region of interest of the subject to be imaged; a first electrode provided on a first surface of the piezoelectric layer; a second electrode provided on a second surface of the piezoelectric layer; a circuit that is electrically connected to the first electrode and the second electrode; and a respective frame, of the frame array, that is provided on at least a third surface of the piezoelectric layer, and that electrically connects the first electrode to the circuit.

### BRIEF DESCRIPTION OF DRAWINGS

FIG. 1 is a diagram of example components of an ultrasound system.

FIG. 2A is a diagram of a transducer of an ultrasound probe including a frame that contacts one of a third surface, a fourth surface, a fifth surface, or a sixth surface of a piezoelectric layer.

FIG. 2B is a diagram of a transducer of an ultrasound probe including a frame that contacts at least two of a third surface, a fourth surface, a fifth surface, or a sixth surface of a piezoelectric layer.

FIG. 2C is a diagram of a transducer with a wrap-around electrode in an azimuth direction.

FIG. 2D is a diagram of a transducer with a wrap-around electrode in an elevation direction.

FIG. 3A is a diagram of a carrier, a frame array, and piezoelectric layers.

FIG. 3B is a diagram of a carrier, a frame array, and piezoelectric layers.

FIG. 4A is a diagram of a method of wafer scale fabrication of ultrasound transducers depicting a carrier and a frame array.

FIG. 4B is a diagram of a method of wafer scale fabrication of ultrasound transducers depicting a carrier, a frame array, and piezoelectric layers.

FIG. 4C is a diagram of a method of wafer scale fabrication of ultrasound transducers depicting a carrier, a frame array, piezoelectric layers, and a grinding plane.

FIG. 4D is a diagram of a method of wafer scale fabrication of ultrasound transducers depicting a carrier, a frame array, piezoelectric layers, and a top layer.

FIG. 4E is a diagram of a method of wafer scale fabrication of ultrasound transducers depicting a carrier, a frame array, piezoelectric layers, a top layer, and singulation planes.

FIG. 4F is a diagram of a method of wafer scale fabrication of ultrasound transducers depicting a singulated acoustic stack used for transducer fabrication.

FIG. 5A is a diagram of a tiling configuration and a singulation configuration for a rectangular piezoelectric layer in which the transducer includes a frame spaced apart in the elevation direction.

FIG. 5B is a diagram of a tiling configuration and a singulation configuration for a rectangular piezoelectric layer in which the transducer includes a frame spaced apart in the azimuth direction.

FIG. 5C is a diagram of a singulation configuration for a square piezoelectric layer without wafer scale fabrication in which the transducer includes a frame spaced apart in the azimuth direction.

FIG. 5D is a diagram of a singulation configuration for a square piezoelectric layer without wafer scale fabrication in which the transducer includes a frame spaced apart in the elevation direction.

FIG. 5E is a diagram of a tiling configuration and a singulation configuration for a square piezoelectric layer with wafer scale fabrication in which the transducer includes a frame spaced apart in the elevation direction.

FIG. 5F is a diagram of a tiling configuration and a singulation configuration for a square piezoelectric layer with wafer scale fabrication in which the transducer includes a frame spaced apart in the azimuth direction.

FIG. 6 is a diagram of an example method for wafer scale fabrication of transducers.

### DETAILED DESCRIPTION

As addressed above, the fabrication of a large number of transducers from single-crystal piezoelectric materials may be inefficient, time-consuming, expensive, difficult, etc., due to the dimensions of the single-crystal piezoelectric materials. Further, as addressed above, conventional approaches of fabricating wrap-around electrodes may result in wrap-around electrodes that are prone to degradation, which can induce signal issues, noise, etc., and negatively affect the reliability and performance of the transducer.

Some embodiments herein provide an ultrasound transducer including a piezoelectric layer, electrodes that are electrically connected to a circuit, and an electrically conductive frame that connects one of the electrodes to the circuit, which thereby forms a wrap-around electrode. Further, the present disclosure relates to a method of wafer scale fabrication of ultrasound transducers using a frame array. In this way, some embodiments herein provide an improvement to the fabrication process of transducers for ultrasound probes by permitting wafer scale fabrication via the usage of a frame array, piezoelectric layers, and one or more common layers which are then singulated to form individual singulated transducers, which increases fabrication efficiency, volume, and throughput. Further, in this way, some embodiments herein provide an improved electrical connection to the top electrode by utilizing a frame of the frame array that remains after singulation of the individual singulated transducers.

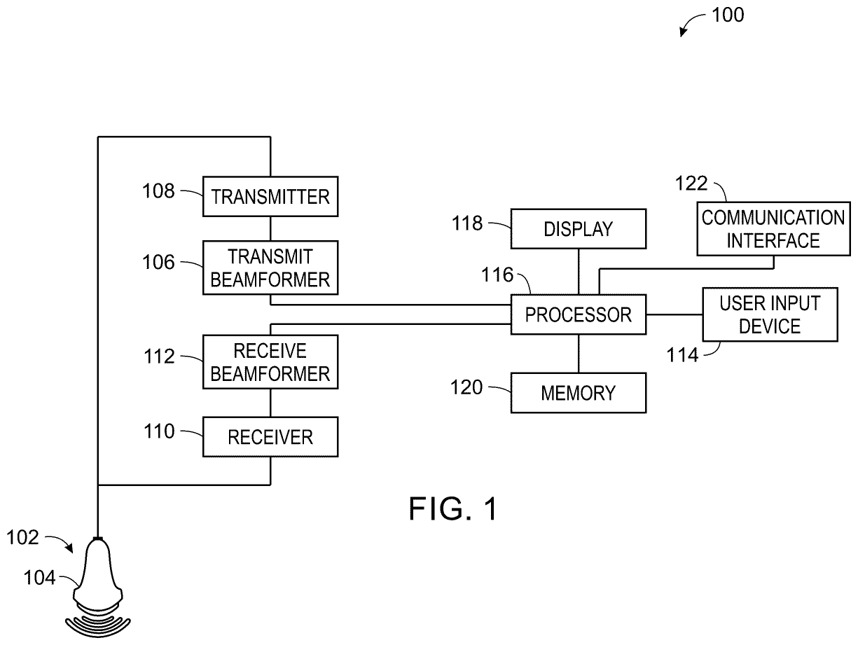

FIG. 1 is a diagram of example components of an ultrasound system 100. As shown in FIG. 1, the ultrasound system 100 may include an ultrasound probe 102, a transducer 104, a transmit beamformer 106, a transmitter 108, a receiver 110, a receive beamformer 112, a user input device 114, a processor 116, a display 118, a memory 120, and a communication interface 122. The foregoing components may be connected via wired or wireless connections.

The ultrasound probe 102 may be configured to acquire ultrasound data for medical imaging, acquire ultrasound data for measuring blood flow, transmit ultrasound signals for tissue ablation, or the like. For example, the ultrasound probe 102 may be a linear probe, a phase array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound probe 102 may be configured to generate ultrasound signals, emit the ultrasound signals towards the region of interest of a subject, receive echo ultrasound signals that are back-scattered from the region of interest of the subject, generate ultrasound data based on the echo ultrasound signals, and output the ultrasound data.

The transmit beamformer 106 may be configured to apply delay times to electrical signals provided to the transducer 104 of the ultrasound probe 102 to focus corresponding ultrasound signals at the region of interest. The transmitter 108 may be configured to transmit electrical signals to the transducer 104 to drive the transducer 104 to emit ultrasound signals towards the region of interest. The transducer 104 may be configured to receive the electrical signals from the transmitter 108, convert the electrical signals into ultrasound signals, and emit the ultrasound signals towards the region of interest. The transducer 104 may be configured to receive echo ultrasound signals that are back-scattered by the region of interest, convert the echo ultrasound signals into electrical signals, and provide the electrical signals to the receiver 110. The receiver 110 may be configured to receive electrical signals from the elements, and provide the electrical signals to the receive beamformer 112. The receive beamformer 112 may apply delay times to the electrical signals received from the transducer 104.

The user input device 114 may be configured to receive a user input, and provide the user input to the processor 116. For example, the user input device 114 may be a touch screen display, a keyboard, a keypad, a mouse, a button, a switch, a microphone, or the like. Additionally, or alternatively, the user input device 114 may be configured to sense information. For example, the user input device 114 may sense information from an electro-magnetic positioning system, an inertial measurement system, an accelerometer, a gyroscope, an actuator, or the like.

The processor 116 may be configured to perform the operations as described herein. For example, the processor 116 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. The processor 116 may be implemented in hardware, firmware, or a combination of hardware and software. The processor 116 may include one or more processors 116 configured to perform the operations described herein. For example, a single processor 116 may be configured to perform all of the operations described herein. Alternatively, multiple processors 116, collectively, may be configured to perform all of the operations described herein, and each of the multiple processors 116 may be configured to perform a subset of the operations descried herein. For example, a first processor 116 may perform a first subset of the operations described herein, a second processor 116 may be configured to perform a second subset of the operations described herein, etc.

The processor 116 may be configured to control the ultrasound probe 102 to acquire ultrasound data. The processor 116 may be configured to control which of elements of the transducer 104 are active, and control the shape of a beam emitted from the transducer 104 of the ultrasound probe 102. The processor 116 may generate ultrasound images for display. For example, the processor 116 may generate B-mode images, color Doppler images, M-mode images, color M-mode images, or the like. The ultrasound images may be 3D images, 2D images, single plane images, bi-plane images, three-plane images, multi-plane images, or the like. The ultrasound images may correspond to various anatomical planes (e.g., sagittal, coronal, and transverse) of the region of interest.

The display 118 may be configured to display information. For example, the display 118 may be a monitor, an LED display, a cathode ray tube, a projector display, a touchscreen, tablet computer, mobile phone, or the like. The display 118 may display ultrasound images based on the ultrasound data in real-time. For example, the display 118 may display the ultrasound images within one second, two seconds, five seconds, etc., of the ultrasound data being acquired by the ultrasound probe 102.

The memory 120 may be configured to store information and/or instructions for use by the processor 116. The memory 120 may be a non-transitory computer-readable medium. For example, the memory 120 may be a random access memory (RAM), a read only memory (ROM), and/or another type of dynamic or static storage device (e.g., a flash memory, a magnetic memory, and/or an optical memory) that stores information and/or instructions for use by the processor 116. The memory 120 may be configured to store instructions that, when executed by the processor 116, cause the processor 116 to perform the operations described herein.

The communication interface 122 may be configured to enable the processor 116 to communicate with other systems, such as via a wired connection, a wireless connection, or a combination of wired and wireless connections. For example, the communication interface 122 may include an Ethernet interface, an optical interface, a coaxial interface, an infrared interface, a radio frequency (RF) interface, a universal serial bus (USB) interface, a Wi-Fi interface, a cellular network interface, or the like.

The number and arrangement of the components of the ultrasound system 100 shown in FIG. 1 are provided as an example. In practice, the ultrasound system 100 may include additional components, fewer components, different components, or differently arranged components than those shown in FIG. 1. Additionally, or alternatively, a set of components (e.g., one or more components) of the ultrasound system 100 may perform one or more functions described as being performed by another set of components of the ultrasound system 100.

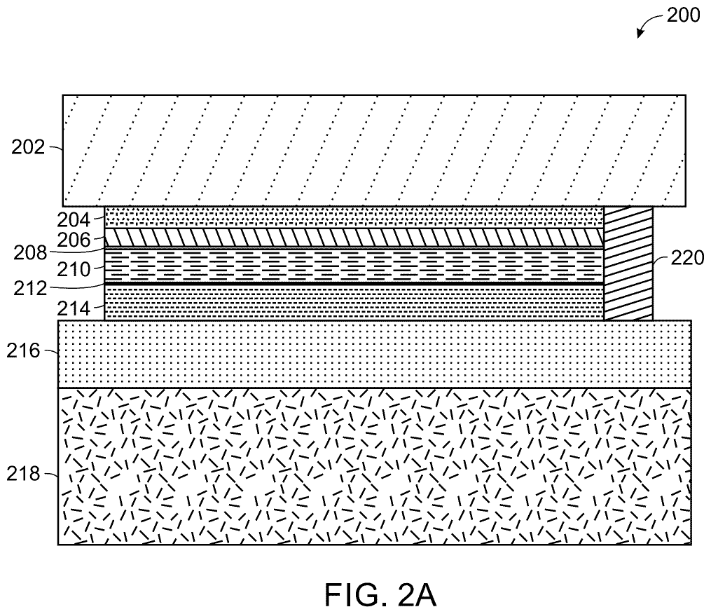

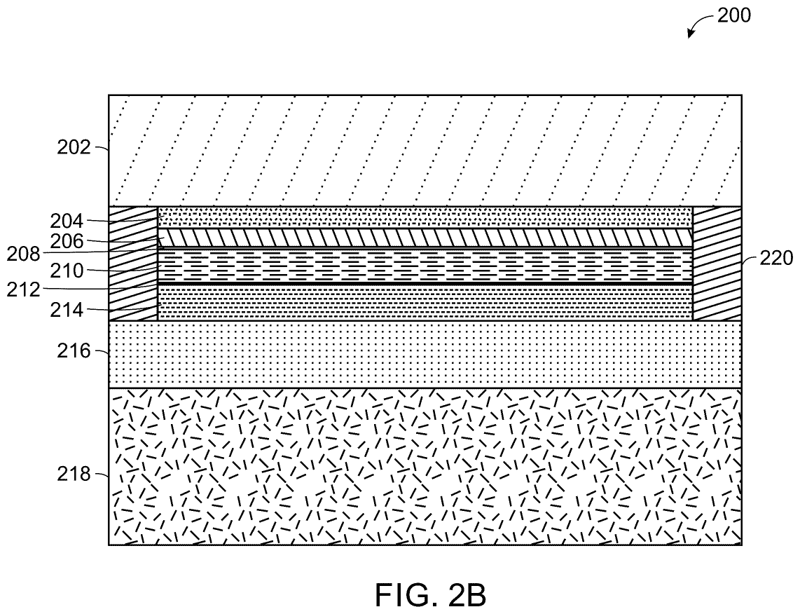

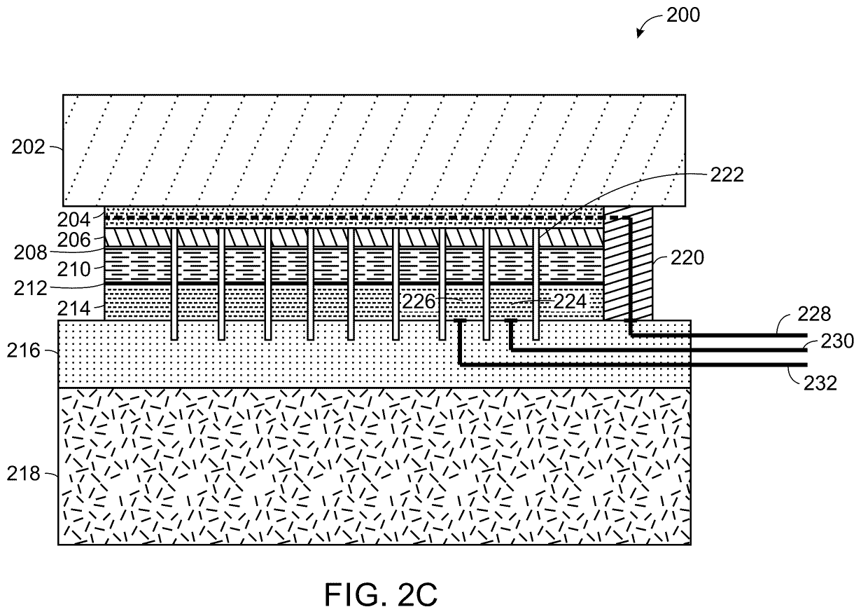

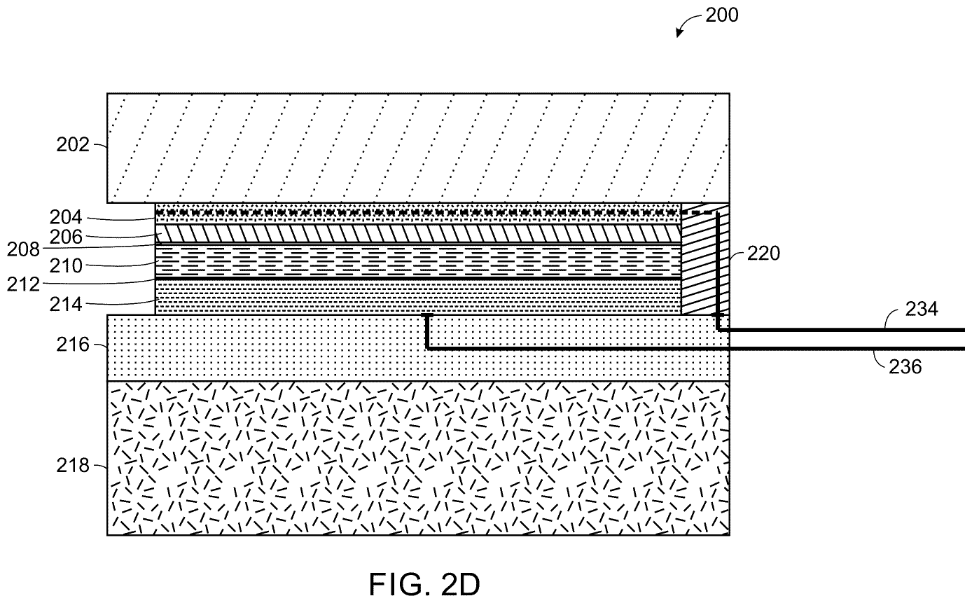

FIG. 2A is a diagram of a transducer 200 of an ultrasound probe including a frame that contacts one of a third surface, a fourth surface, a fifth surface, or a sixth surface of a piezoelectric layer. As shown in FIG. 2A, the transducer 200 may include a lens 202, a second acoustic matching layer 204, a first acoustic matching layer 206, a first electrode 208, a piezoelectric layer 210, a second electrode 212, an acoustic dematching layer 214, a circuit 216, a backing layer 218, and a frame 220.

According to an embodiment, the lens 202 may be configured to direct an ultrasound signal towards the region of interest of the subject. For example, the lens 202 may be silicone, epoxy, rubber, or the like. According to an embodiment, the second acoustic matching layer 204 and/or the first acoustic matching layer 206 may be configured to facilitate matching of an impedance differential that may exist between the relatively high impedance transducer 200 and the relatively low impedance subject. For example, the second acoustic matching layer 204 and/or the first acoustic matching layer 206 may be graphite, plastic, resin, or the like. According to an embodiment, the first acoustic matching layer 206 may be configured to facilitate matching of an impedance differential that may exist between the piezoelectric layer 210 and the second acoustic matching layer 204, and the second acoustic matching layer 204 may be configured to facilitate matching of an impedance differential that may exist between the first acoustic matching layer 206 and the subject.

According to an embodiment, the first electrode 208 may be a signal electrode, and the second electrode 212 may be a ground electrode that are configured to contact the piezoelectric layer 210 to transmit electrical signals. Alternatively, the first electrode 208 may be a ground electrode, and the second electrode 212 may be a signal electrode that are configured to contact the piezoelectric layer 210. For example, the first electrode 208 and/or the second electrode 212 may be gold, copper, nickel, silver, chromium, aluminum, or the like.

According to an embodiment, the piezoelectric layer 210 may be configured to receive an electrical signal, deform based on the electrical signal, generate an ultrasound signal based on the deformation, and transmit the ultrasound signal towards a region of interest.

Additionally, or alternatively, the piezoelectric layer 210 may be configured to receive an echo signal reflected by the region of interest, deform based on the echo signal, generate an electrical signal based on the deformation, and transmit the electrical signal. For example, the piezoelectric layer 210 may be Pb(Mg_{1/3}Nb_{2/3})O₃-PbTiO₃ (“PMN-PT”), Pb(In_{1/2}Nb_{1/2})O₃-Pb(Mg_{1/3}Nb_{2/3})O₃-PbTiO₃ (“PIN-PMN-PT”), Pb(ZrTi) (“PZT”), or the like.

According to an embodiment, the acoustic dematching layer 214 may be configured to decrease insertion losses and enhance a frequency bandwidth of the transducer 200. For example, the acoustic dematching layer 214 may be tungsten carbide, silicon carbide, or the like. According to an embodiment, the circuit 216 may be configured to electrically connect to the first electrode 208 and connect to the second electrode 212 to enable application of a voltage to induce deformation of each of the elements of the piezoelectric layer 210. For example, the circuit may be an ASIC, a PCB, a flexible circuit, or the like. The circuit 216 may electrically connect to the transmitter 108 and the receiver 110. According to an embodiment, the backing layer 218 may be configured to attenuate ultrasound signals directed from the piezoelectric layer 210 in a direction opposite to the subject, and attenuate ultrasound signals deflected by a housing of the ultrasound probe 102. According to an embodiment, the frame 220 may be configured to electrically connect to the first electrode 208. For example, the frame 220 may be an electrically conductive material, such as graphite, metal, Polyimide, or the like.

According to an embodiment, the piezoelectric layer 210 may include a first surface, a second surface, a third surface, and a fourth surface. The first electrode 208 may be provided on the first surface of the piezoelectric layer 210. The second electrode 212 may be provided on the second surface of the piezoelectric layer 210. The frame 220 may be provided on one or more of a third surface of the piezoelectric layer 210, a fourth surface of the piezoelectric layer 210, a fifth surface of the piezoelectric layer 210, and a sixth surface of the piezoelectric layer 210. For example, as shown in FIG. 2A, the frame 220 may be provided on a third surface of the piezoelectric layer 210. The third surface may be an end surface in an x-axis (azimuth) direction or a y-axis (elevation) direction.

As shown in FIG. 2A, the lens 202 may be provided in an uppermost position in the z-axis (propagation) direction. The second acoustic matching layer 204 may be provided below the lens 202. The first acoustic matching layer 206 may be provided below the second acoustic matching layer 204. The first electrode 208 may be provided below the first acoustic matching layer 206. The piezoelectric layer 210 may be provided below the first electrode 208. The second electrode 212 may be provided below the piezoelectric layer 210. The acoustic dematching layer 214 may be provided below the second electrode 212. The circuit 216 may be provided below the acoustic dematching layer 214. The backing layer 218 may be provided below the circuit 216. The frame 220 may extend between the lens 202 and the circuit 216. Further, the frame 220 may contact respective side surfaces of the second acoustic matching layer 204, the first acoustic matching layer 206, the first electrode 208, the piezoelectric layer 210, the second electrode 212, and the acoustic dematching layer 214.

FIG. 2B is a diagram of a transducer 200 of an ultrasound probe including a frame that contacts at least two of a third surface, a fourth surface, a fifth surface, or a sixth surface of a piezoelectric layer. As shown in FIG. 2B, the frame 220 may be provided on a third surface of the piezoelectric layer 210 and a fourth surface of the piezoelectric layer 210. The third surface and the fourth surface may be end surfaces in an x-axis (azimuth) direction or a y-axis (elevation) direction.

FIG. 2C is a diagram of a transducer 200 with a wrap-around electrode in an azimuth direction. As shown in FIG. 2C, the frame 220 may be provided on a third surface of the piezoelectric layer 210. The third surface may be an end surface in the x-axis (azimuth) direction. As shown in FIG. 2C, the transducer 200 may include kerfs 222. The kerfs 222 may be provided in the first acoustic matching layer 206, the first electrode 208, the piezoelectric layer 210, the second electrode 212, the acoustic dematching layer 214, and the circuit 216 along the y-axis direction and spaced apart along the x-axis (azimuth) direction via dicing in order to provide electrically-insulated elements. For example, as shown, the transducer 200 may include a first element 224 and a second element 226. The kerfs 222 may be filled with an electrically non-conductive or insulating material (e.g., silicone). As shown in FIG. 2C, the kerfs 222 may extend entirely through the first acoustic matching layer 206, the first electrode 208, the piezoelectric layer 210, the second electrode 212, the acoustic dematching layer 214, and may extend partially through the circuit 216. Although two elements 224 and 226 are labelled in FIG. 2C, it should be understood that the transducer 200 may include any number of elements that are provided in a 1D or 2D array of elements. As further shown in FIG. 2C, a ground signal line 228 may be provided in the second acoustic matching layer 204, the frame 220, and the circuit 216. Further, as shown in FIG. 2C, a first signal line may 230 may be provided in the first element 224 and the circuit 216. Further, as shown in FIG. 2C, a second signal line 232 may be provided in the second element 226 and the circuit 216. In this way, the frame 220 may electrically connect the first electrode 208 that is provided above the piezoelectric layer 210 in the x-axis direction to the circuit 216 that is provided below the piezoelectric layer 210 in the x-axis direction to form a wrap-around electrode in the azimuth direction.

FIG. 2D is a diagram of a transducer 200 with a wrap-around electrode in an elevation direction. As shown in FIG. 2D, the frame 220 may be provided on a third surface of the piezoelectric layer 210. The third surface may be an end surface in the y-axis (elevation) direction. As further shown in FIG. 2D, a ground signal line 234 may be provided in the second acoustic matching layer 204, the frame 220, and the circuit 216. Further, as shown in FIG. 2D, a signal line may 236 may be provided in the first element 224 and the circuit 216.

Although FIGS. 2A-2D depict particular components and a particular arrangement of components, it should be understood that other embodiments may include other components, less components, additional components, etc., and/or may include a different arrangement of components.

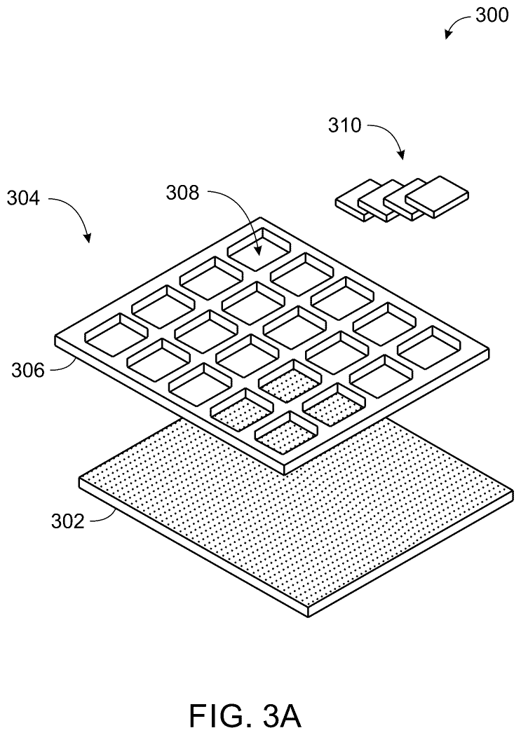

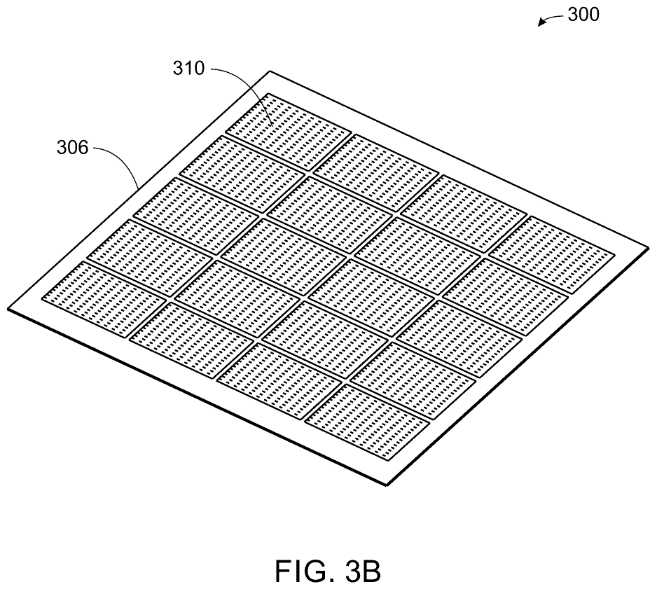

FIG. 3A is a diagram 300 of a carrier 302, a frame array 304, and piezoelectric layers 310, and FIG. 3B is a diagram 300 of a carrier 302, a frame array 304, and piezoelectric layers 310. As shown in FIG. 3A, the carrier 302 may be provided to support the frame array 304. The carrier 302 may be the acoustic dematching layer 214 as shown in FIGS. 2A-2D, the first acoustic matching layer 206 as shown in FIGS. 2A-2D, a substrate that is used during the wafer scale fabrication process and that is removed before the transducer 200 is provided in the ultrasound probe 102, or the like. The frame array 304 may include a frame 306 and slots 308.

The frame array 304 may be an n×n array of slots 308, an m×n array of slots 308, or the like. The piezoelectric layers 310 may include corresponding shapes and dimensions as the slots 308, and may be respectively provided in respective slots 308 of the frame array 304, as shown in FIG. 3B.

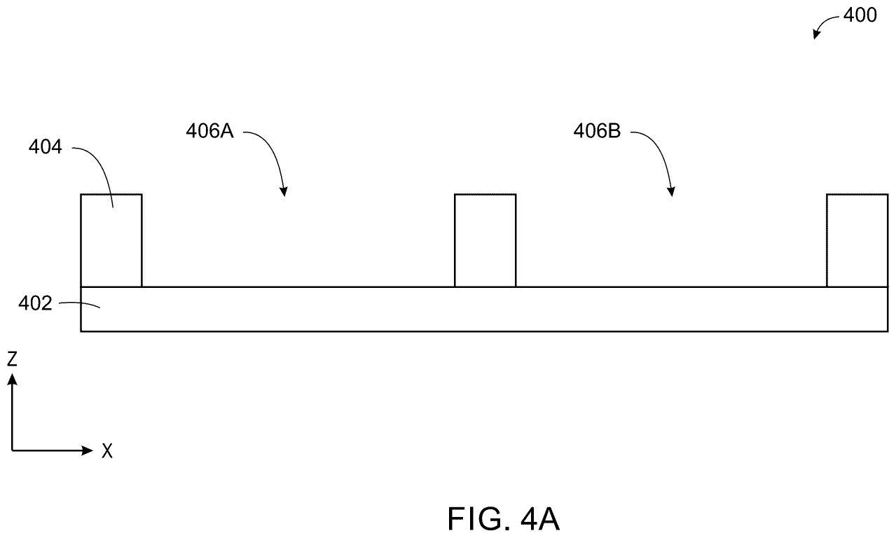

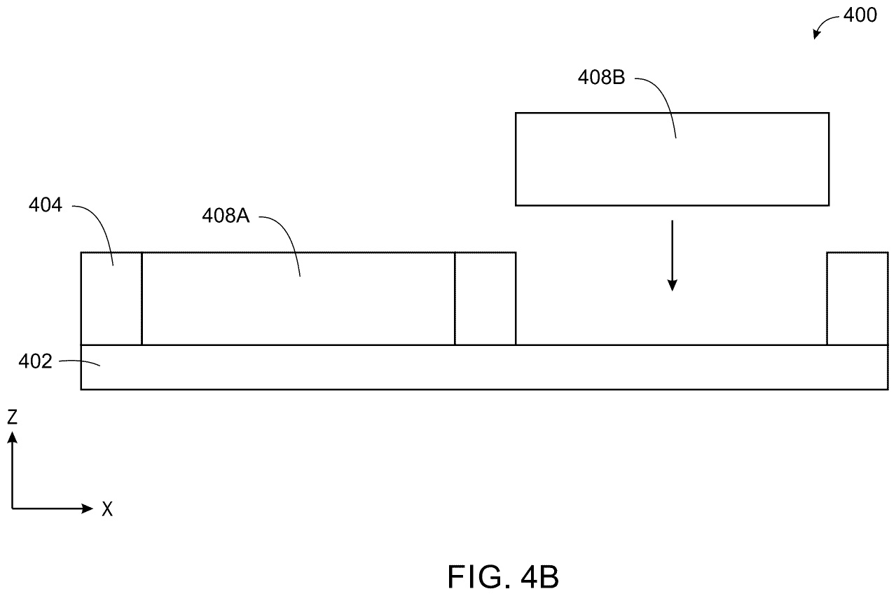

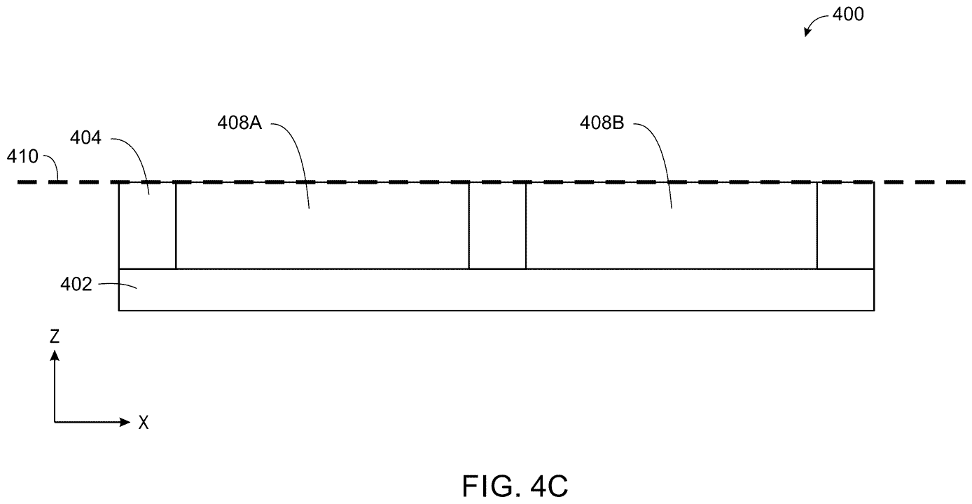

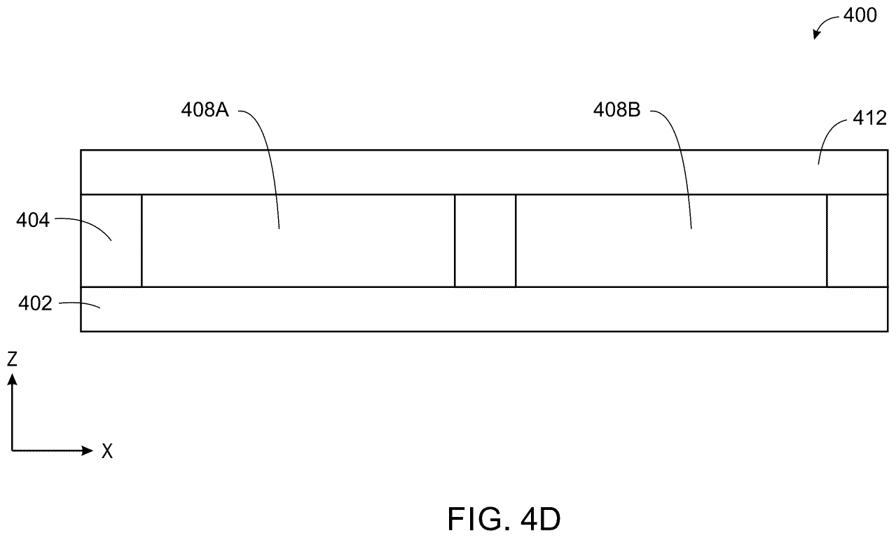

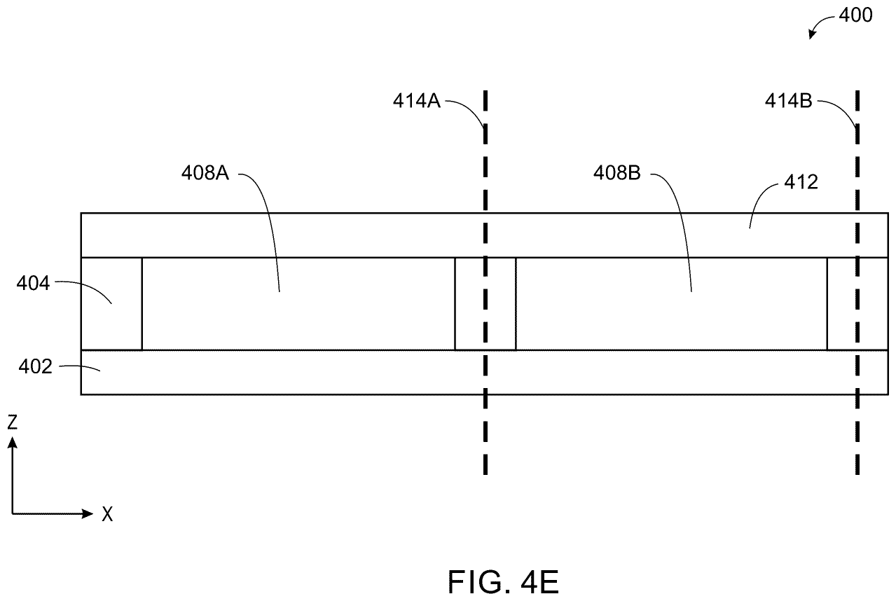

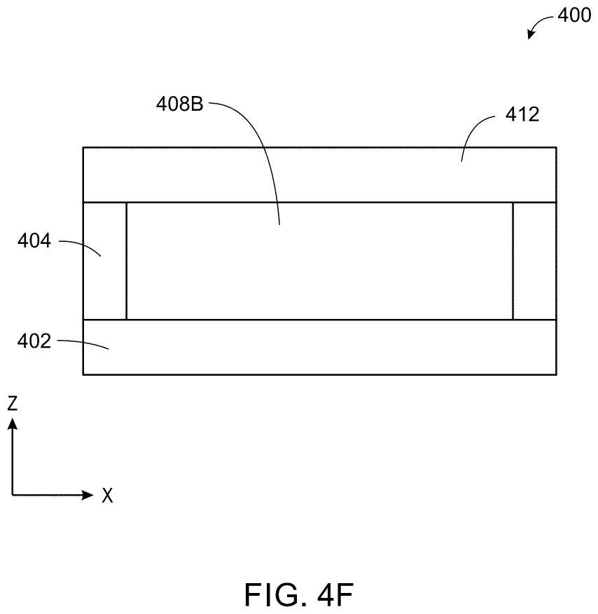

FIG. 4A is a diagram of a method 400 of wafer scale fabrication of ultrasound transducers 200 depicting a carrier 402 and a frame array 404. As shown in FIG. 4A, the carrier 402 may include a top surface that extends along the x-axis, a bottom surface that extends along the x-axis and that is spaced apart from the top surface along the z-axis, a left surface that extends along the z-axis, and a right surface that extends along the z-axis and that is spaced apart from the left surface along the x-axis. As further shown in FIG. 4A, the frame array 404 may include a top surface that extends along the x-axis, and a bottom surface that extends along the x-axis and that is spaced apart from the top surface along the z-axis. Further, the frame array 404 may include a first slot 406A and a second slot 406B. Although two slots are shown in FIG. 4A, it should be understood that the frame array 404 may include n slots arranged along the x-axis direction, and n slots arranged along the y-axis direction to form an n×n array of slots, may include n slots arranged along the x-axis direction, and m slots arranged along the y-axis direction to form an n×m array of slots, or may include m slots arranged along the x-axis direction, and n slots arranged along the y-axis direction to form an m×n array of slots. The frame array 404 may be provided on the carrier 402, such that the bottom surface of the frame array 404 contacts the top surface of the carrier 402. The frame array 404 may be bonded to the carrier 402 using an adhesive (e.g., an epoxy resin).

FIG. 4B is a diagram of a method 400 of wafer scale fabrication of ultrasound transducers depicting a carrier 402, a frame array 404, and piezoelectric layers 408. As shown in FIG. 4B, each of the first piezoelectric layer 408A and the second piezoelectric layer 408B may include respective top surfaces that extend along the x-axis, and bottom surfaces that extend along the x-axis and that are spaced apart from the top surfaces along the z-axis. Further, each of the first piezoelectric layer 408A and the second piezoelectric layer 408B may include respective left surfaces that extend along the z-axis, and right surfaces that extend along the z-axis and that are spaced apart from the left surfaces along the x-axis. The first piezoelectric layer 408A may be provided in the first slot 406A, such that the bottom surface of the first piezoelectric layer 408A contacts the top surface of the carrier 402, the left surface contacts an internal surface of the frame array 404 in the first slot 406A, and the right surface contacts an internal surface of the frame array 404 in the first slot 406A. The first piezoelectric layer 408A may be bonded to the frame array 404 and/or the carrier 402 via an adhesive. The second piezoelectric layer 408B may be provided in the second slot 406B, such that the bottom surface of the second piezoelectric layer 408B contacts the top surface of the carrier 402, the left surface contacts an internal surface of the frame array 404 in the second slot 406B, and the right surface contacts an internal surface of the frame array 404 in the second slot 406B. The second piezoelectric layer 408B may be bonded to the frame array 404 and/or the carrier 402 via an adhesive.

FIG. 4C is a diagram of a method 400 of wafer scale fabrication of ultrasound transducers depicting a carrier 402, a frame array 404, piezoelectric layers 408, and a grinding plane 410. As shown in FIG. 4C, after the first piezoelectric layer 408A and the second piezoelectric layer 408B are provided in the frame array 404, the respective top surfaces of the first piezoelectric layer 408A, the second piezoelectric layer 408B, and the frame array 404 may form a collective top surface. The method 400 may include a grinding step in order to provide a leveled top surface, and/or to remove any excess adhesive. The respective top surfaces of the first piezoelectric layer 408A, the second piezoelectric layer 408B, and the frame array 404 may be ground along a grinding plane 420.

FIG. 4D is a diagram of a method 400 of wafer scale fabrication of ultrasound transducers 200 depicting a carrier 402, a frame array 404, piezoelectric layers 408, and a top layer 412. As shown in FIG. 4D, a top layer 412 may include a top surface that extends along the x-axis, a bottom surface that extends along the x-axis and that is spaced apart from the top surface along the z-axis, a left surface that extends along the z-axis, and a right surface that extends along the z-axis and that is spaced apart from the left surface along the x-axis. The top layer 412 may be the acoustic dematching layer 214 as shown in FIGS. 2A-2D, the first acoustic matching layer 206 as shown in FIGS. 2A-2D, a substrate that is used during the wafer scale fabrication process and that is removed before the transducer 200 is provided in the ultrasound probe 102, or the like. For example, if the carrier 402 is the acoustic dematching layer 214, then the top layer 412 may be the first acoustic matching layer 206. As another example, if the carrier 402 is the first acoustic matching layer 206, then the top layer 412 may be the acoustic dematching layer 214. The bottom surface of the top layer 412 may contact the top surface of the frame array 404, the top surface of the first piezoelectric layer 408A, and the top surface of the second piezoelectric layer 408B.

FIG. 4E is a diagram of a method 400 of wafer scale fabrication of ultrasound transducers 200 depicting a carrier 402, a frame array 404, piezoelectric layers 408, a top layer 412, and singulation planes 414, and FIG. 4F is a diagram of a method 400 of wafer scale fabrication of ultrasound transducers 200 depicting a singulated transducer 200. As shown in FIG. 4E, the carrier 402, the frame array 404, the piezoelectric layers 408, and the top layer 412 may be singulated along singulations planes 414A and 414B to form a singulated transducer 200 as shown in FIG. 4F.

A signal electrode and a ground electrode may be provided on the piezoelectric layer 408 to yield a transducer 200 as shown in FIGS. 2A-2D. In this way, the remaining portions of the frame array 404 after singulation may electrically connect the top electrode, thereby providing an improved electrical connection and improving reliability of the transducer 200. Further, in this way, the method 400 provides wafer scale fabrication of ultrasound transducers 104 by permitting an array of transducers 104 to be fabricated simultaneously using a frame array 404 and one or more common layers (e.g., the carrier 402 and the top layer 412), and then singulated to result in singulated transducers 104. In this way, the wafer scale fabrication may increase efficiency of fabrication and throughput of fabrication by utilizing common layers and/or by reducing the need to metallize the side surfaces of the piezoelectric layer 408.

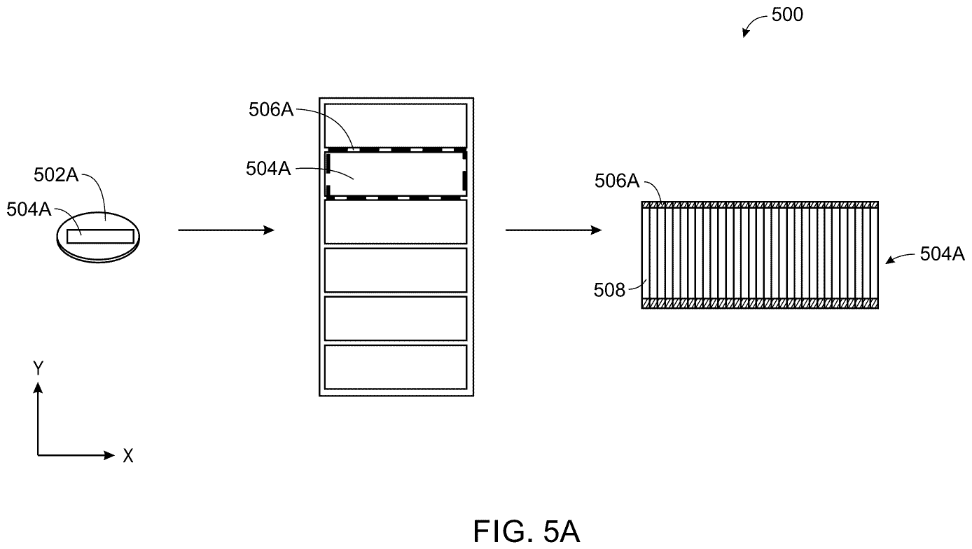

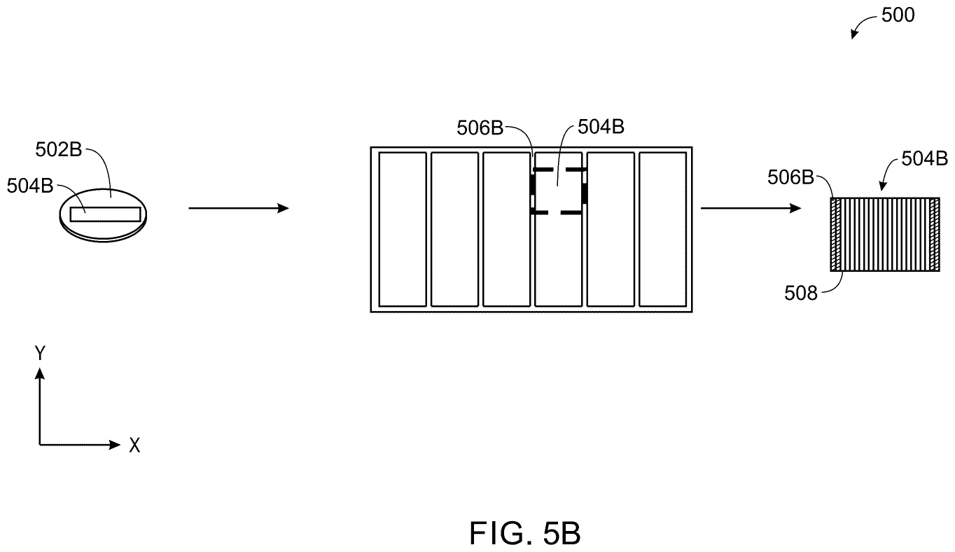

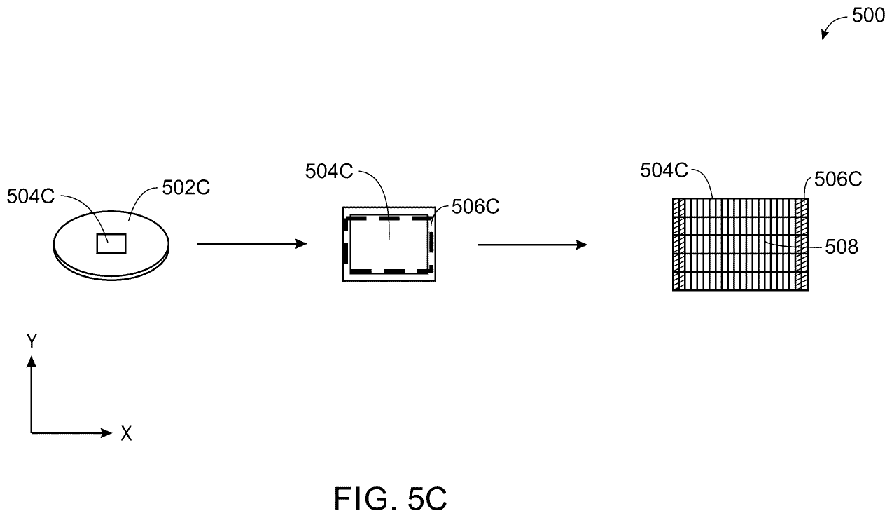

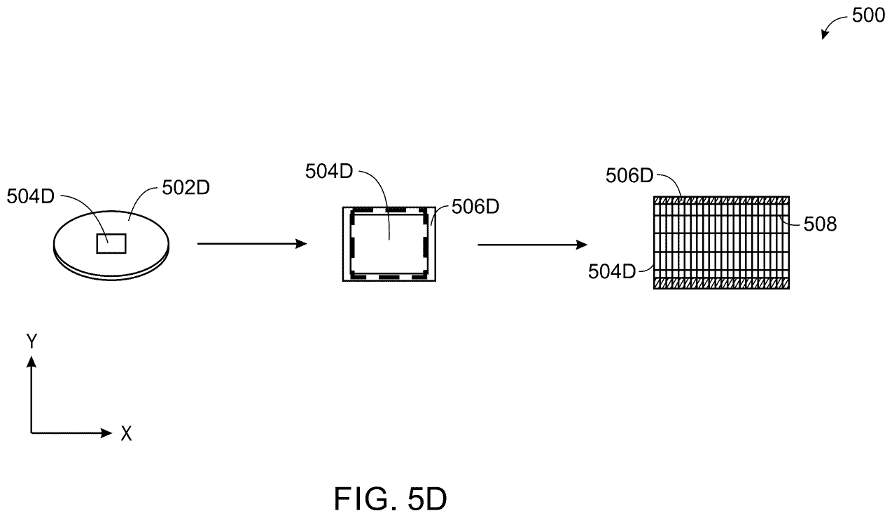

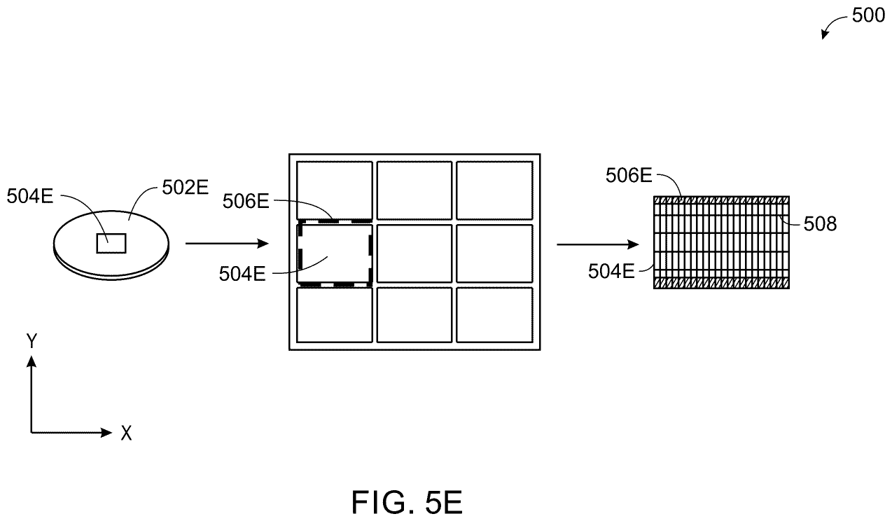

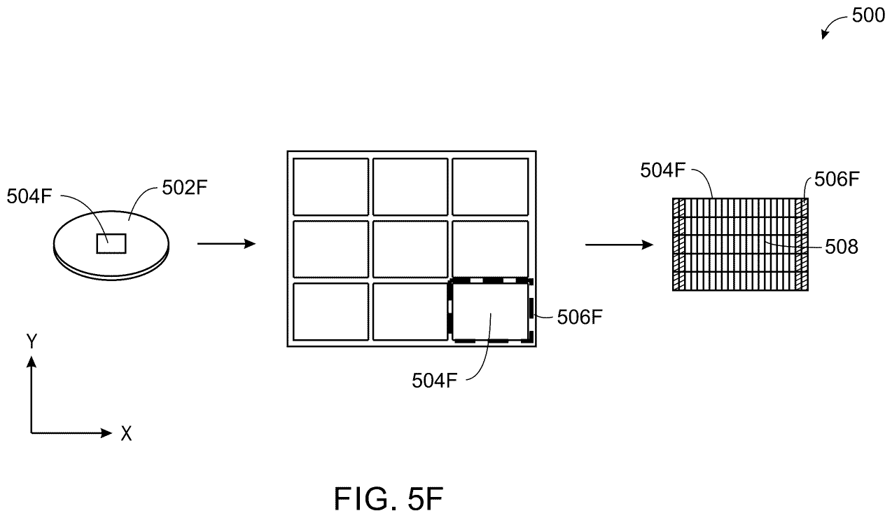

FIG. 5A is a diagram 500 of a tiling configuration and a singulation configuration for a rectangular piezoelectric layer 504A in which the transducer 200 includes a frame 506A spaced apart in the elevation direction. As shown in FIG. 5A, the piezoelectric layer 504A may be formed from a slice 502A of an ingot (or boule). The piezoelectric layer 504A may be provided in a frame 506A. The piezoelectric layer 504A may be longer in an x-axis direction (e.g., azimuth direction) as compared to a y-axis direction (e.g., elevation direction). Similarly, slots of the frame 506A may be longer in the x-axis direction than as compared to the y-axis direction. The piezoelectric layers 506A may be provided in the frame array 506A in accordance with a tiling configuration in which the piezoelectric layers 506A are spaced apart along the y-axis direction. Further, the frame array 506A and the piezoelectric layer 506A may be singulated in accordance with a singulation configuration to form a singulated transducer 200. The singulation configuration renders frames 506A of the frame array that are spaced apart along the y-axis direction. Kerfs 508 may be provided in the piezoelectric layer 504A along the y-axis direction and spaced apart along the x-axis direction via dicing in order to provide electrically-insulated elements forming a 1-dimensional array of transducer elements. The kerfs 508 may be filled with an electrically non-conductive or insulating material (e.g., silicone).

FIG. 5B is a diagram 500 of a tiling configuration and a singulation configuration for a rectangular piezoelectric layer 504B in which the transducer 200 includes a frame 506B spaced apart in the azimuth direction. As shown in FIG. 5B, the piezoelectric layer 504B may be formed from a slice 502B of an ingot (or boule). The piezoelectric layer 504B may be provided in a frame 506B. The piezoelectric layer 504B may be longer in a y-axis direction (e.g., elevation direction) as compared to an x-axis direction (e.g., azimuth direction). Similarly, slots of the frame 506B may be longer in the y-axis direction than as compared to the x-axis direction. The piezoelectric layers 506B may be provided in the frame array 506B in accordance with a tiling configuration in which the piezoelectric layers 506B are spaced apart along the x-axis direction. Further, the frame array and the piezoelectric layer 506B may be singulated in accordance with a singulation configuration to form a singulated transducer 200. The singulation configuration renders frames 506B of the frame array that are spaced apart along the x-axis direction. Kerfs 508 may be provided in the piezoelectric layer 504B along the y-axis direction and spaced apart along the x-axis direction and along the x-axis direction and spaced apart along the y-axis direction via dicing in order to provide electrically-insulated elements forming a 2-dimensional array of transducer elements. The kerfs 508 may be filled with an electrically non-conductive or insulating material (e.g., silicone).

FIG. 5C is a diagram of a singulation configuration for a square piezoelectric layer 504B without wafer scale fabrication in which the transducer 200 includes a frame 506C spaced apart in the azimuth direction. As shown in FIG. 5C, the piezoelectric layer 504C may be formed from a slice 502C of an ingot (or boule). The piezoelectric layer 504C may be provided in a frame 506C. The piezoelectric layer 504C may be substantially the same length in an x-axis direction (e.g., azimuth direction) as compared to a y-axis direction (e.g., elevation direction).

The frame 506C and the piezoelectric layer 504C may be singulated in accordance with a singulation configuration to form a singulated transducer 200. The singulation configuration renders the frame 506C that is spaced apart along the x-axis direction. Kerfs 508 may be provided in the piezoelectric layer 504C along the y-axis direction and spaced apart along the x-axis direction and along the x-axis direction and spaced apart along the y-axis direction via dicing in order to provide electrically-insulated elements forming a 2-dimensional array of transducer elements. The kerfs 508 may be filled with an electrically non-conductive or insulating material (e.g., silicone).

FIG. 5D is a diagram 500 of a singulation configuration for a square piezoelectric layer 504D without wafer scale fabrication in which the transducer 200 includes a frame 506D spaced apart in the elevation direction. As shown in FIG. 5D, the piezoelectric layer 504D may be formed from a slice 502D of an ingot (or boule). The piezoelectric layer 504D may be provided in a frame 506D. The piezoelectric layer 504D may be substantially the same length in an x-axis direction (e.g., azimuth direction) as compared to a y-axis direction (e.g., elevation direction). The frame 506D and the piezoelectric layer 504D may be singulated in accordance with a singulation configuration to form a singulated transducer 200. The singulation configuration renders the frame 506D that is spaced apart along the y-axis direction. Kerfs 508 may be provided in the piezoelectric layer 504D along the y-axis direction and spaced apart along the x-axis direction and along the x-axis direction and spaced apart along the y-axis direction via dicing in order to provide electrically-insulated elements forming a 2-dimensional array of transducer elements. The kerfs 508 may be filled with an electrically non-conductive or insulating material (e.g., silicone).

FIG. 5E is a diagram 500 of a tiling configuration and a singulation configuration for a square piezoelectric layer 504E with wafer scale fabrication in which the transducer 200 includes a frame 506E spaced apart in the elevation direction. As shown in FIG. 5E, the piezoelectric layer 504E may be formed from a slice 502E of an ingot (or boule). The piezoelectric layer 504E may be provided in a frame 506E. The piezoelectric layer 504E may be substantially the same length in a y-axis direction (e.g., elevation direction) as compared to an x-axis direction (e.g., azimuth direction). Similarly, slots of the frame 506B may be substantially the same length in the y-axis direction than as compared to the x-axis direction. The piezoelectric layers 506E may be provided in the frame array in accordance with a tiling configuration in which the piezoelectric layers 506E are spaced apart along the x-axis direction and are spaced apart along the y-axis direction. In other words, the frame array 506E is a 2D array. Further, the frame array and the piezoelectric layer 504E may be singulated in accordance with a singulation configuration to form a singulated transducer 200. The singulation configuration renders frames 506E of the frame array that are spaced apart along the y-axis direction. Kerfs 508 may be provided in the piezoelectric layer 504E along the y-axis direction and spaced apart along the x-axis direction via dicing in order to provide electrically-insulated elements forming a 2-dimensional array of transducer elements. The kerfs 508 may be filled with an electrically non-conductive or insulating material (e.g., silicone).

FIG. 5F is a diagram 500 of a tiling configuration and a singulation configuration for a square piezoelectric layer 504F with wafer scale fabrication in which the transducer includes a frame 506F spaced apart in the azimuth direction. As shown in FIG. 5F, the piezoelectric layer 504F may be formed from a slice 502F of an ingot (or boule). The piezoelectric layer 504F may be provided in a frame 506F. The piezoelectric layer 504F may be substantially the same length in a y-axis direction (e.g., elevation direction) as compared to an x-axis direction (e.g., azimuth direction). Similarly, slots of the frame 506F may be substantially the same length in the y-axis direction than as compared to the x-axis direction. The piezoelectric layers 506F may be provided in a frame array in accordance with a tiling configuration in which the piezoelectric layers 506F are spaced apart along the x-axis direction and are spaced apart along the y-axis direction. In other words, the frame array is a 2D array. Further, the frame array and the piezoelectric layer 506F may be singulated in accordance with a singulation configuration to form a singulated transducer 200. The singulation configuration renders frames 506F of the frame array that are spaced apart along the x-axis direction. Kerfs 508 may be provided in the piezoelectric layer 504E along the y-axis direction and spaced apart along the x-axis direction and along the x-axis direction and spaced apart along the y-axis direction via dicing in order to provide electrically-insulated elements forming a 2-dimensional array of transducer elements. The kerfs 508 may be filled with an electrically non-conductive or insulating material (e.g., silicone).

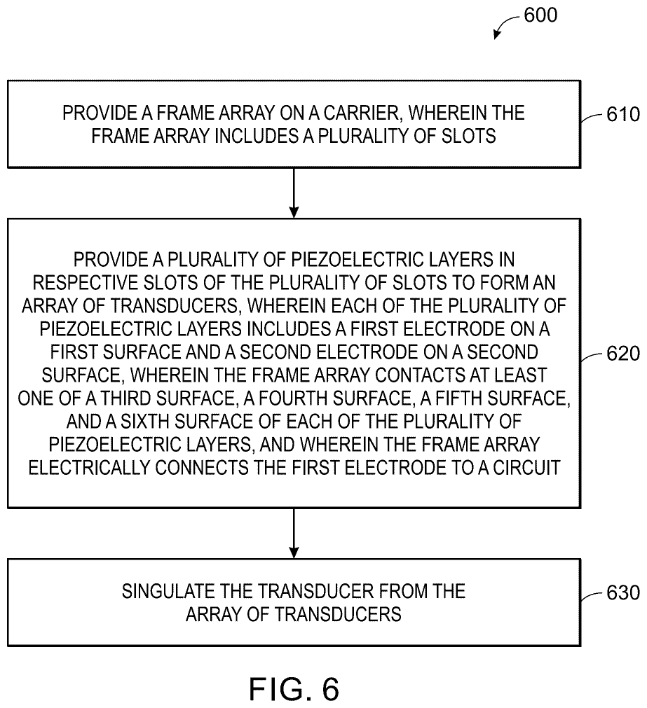

FIG. 6 is a diagram of an example method 600 for wafer scale fabrication of transducers 200. As shown in FIG. 6, the method 600 may include providing a frame array on a carrier, wherein the frame array includes a plurality of slots (operation 610), providing a plurality of piezoelectric layers in respective slots of the plurality of slots to form an array of transducers, wherein each of the plurality of piezoelectric layers includes a first electrode on a first surface and a second electrode on a second surface, wherein the frame array contacts at least one of a third surface, a fourth surface, a fifth surface, and a sixth surface of each of the plurality of piezoelectric layers, and wherein the frame array electrically connects the first electrode to a circuit (operation 620), and singulating the transducer from the array of transducers (operation 630).

According to an embodiment, the transducer 200 may be formed to include a lens 202, a second acoustic matching layer 204, a first acoustic matching layer 206, a first electrode 208, a piezoelectric layer 210, a second electrode 212, an acoustic dematching layer 214, a circuit 216, a backing layer 218, and a frame 220, as shown in FIGS. 2A-2D and/or FIGS. 4A-4F.

The frame array 404 may be provided on the carrier 402 such that a bottom surface of the frame array 404 is provided on a top surface of the carrier 402. The carrier 402 may be the acoustic dematching layer 214 as shown in FIGS. 2A-2D, the first acoustic matching layer 206 as shown in FIGS. 2A-2D, a substrate that is used during the wafer scale fabrication process and that is removed before the transducer 200 is provided in the ultrasound probe 102, or the like.

The frame array 404 may be bonded to the carrier 402 using an adhesive (e.g., an epoxy resin). The frame array 404 may be machined out of any material sheets (e.g., graphite sheets, metal sheets, polymer sheets, etc.). Different machining process may be used such as milling, laser cutting, electrical discharge machining (EDM), laser microjet, etc. depending on the alignment precision requirements. According to an embodiment, a precision of ±10 micrometer may be implemented in the machining of the frame array 404, to achieve an alignment of ±10 micrometer of the piezoelectric layers 408. This precision may be implemented to ensure, or improve, the sub-sequent process steps, for example dicing and singulation of the elements of the transducer 200.

The piezoelectric layers 408 may be provided in respective slots 406 of the frame array 404 to form an array of transducers 200. In this way, respective top surfaces of the piezoelectric layers 408 and the frame array 404 may form a collective top surface, and respective bottom surfaces of the piezoelectric layers 408 and the frame array 404 may form a collective bottom surface that is provided on, and supported by, the carrier 402.

The piezoelectric layers 408 and the frame array 404 may be grinded to level the collective top surface of the piezoelectric layers 408 and the frame array 404 and/or remove excess adhesive.

A first electrode 208 and a second electrode 212 may be provided on respective top surfaces of each of the piezoelectric layers 408 and respective bottom surfaces of each of the piezoelectric layers 408, respectively. The first electrode 208 may be a signal electrode and the second electrode 212 may be a ground electrode, or vice versa. The first electrode 208 and/or the second electrode 212 may be provided on the piezoelectric layers 408 via sputtering, electro-deposition, plating, controlled dispensing, jetting, evaporating, or the like.

A top layer 412 may be provided on the collective top surface of the piezoelectric layers 408 and the frame array 404. The top layer 412 may be the acoustic dematching layer 214 as shown in FIGS. 2A-2D, the first acoustic matching layer 206 as shown in FIGS. 2A-2D, a substrate that is used during the wafer scale fabrication process and that is removed before the transducer 200 is provided in the ultrasound probe 102, a backing layer, or the like. For example, if the carrier 402 is the acoustic dematching layer 214, then the top layer 412 may be the first acoustic matching layer 206. As another example, if the carrier 402 is the first acoustic matching layer 206, then the top layer 412 may be the acoustic dematching layer 214. If the carrier 402 is a substrate, then the carrier 402 may be removed, and the acoustic dematching layer 214 as shown in FIGS. 2A-2D, the first acoustic matching layer 206, or another type of layer, may be provided in place of the carrier 402.

The array of transducers 200 may be singulated to form singulated transducers. Portions of the frame array 404 may remain on each singulated transducer 200 after singulation. One or more portions of the frame array 404 may contact one or more side surfaces of the piezoelectric layer 408 of the singulated transducer to electrically connect the first electrode 208. The one or more portions may be spaced apart in the azimuth direction or the elevation direction. As used herein, “singulate,” “singulated,” “singulation,” etc. may refer to the physical separation of an individual transducer 200 from the transducer array. Singulation may include laser singulation, scribe and break, and dice before grind. Each of the transducers 200 may be physically separated as a result of the singulation, and each transducer 200 may be installed on their individual circuit 216 (e.g., PCB, flexible circuit, etc.) in the ultrasound probe 102.

The singulated transducers may be diced to form kerfs 508 as shown in FIGS. 5A-5F. The kerfs 508 may be spaced apart in one or more of the x-axis direction and the y-axis direction to form a 1D array of elements of the singulated transducer 200 or a 2D array of elements of the singulated transducer 200. The kerfs 508 may be filled with an electrically non-conductive or insulating material (e.g., silicone). Alternatively, the kerfs 508 may not be filled with any material, yet still provide electrical insulation. As used herein, “dicing,” “dice,” diced,” etc., may refer to the cutting of a cavity or slot into one or more layers of the transducer 200. The kerfs 508 may extend entirely through one or more layers (e.g., the second acoustic matching layer 204, the first acoustic matching layer 206, the piezoelectric layer 210, and the acoustic dematching layer 214), and may partially extend through the circuit 216.

The elements may be arranged in a variety of configuration such as 1D arrays, 2D arrays, 1D linear arrays, 2D square arrays, 2D rectangular arrays, 2D annular arrays, or the like. Electrical connections to the elements of the singulated transducer 200 may be formed using a flexible printed circuit, or the like. One or more other layers or components may be added to the transducer 200, such as a lens, a backing, or the like. The singulated transducer 200 may be provided in an ultrasound probe 102 to perform ultrasound applications, such as ultrasound imaging, ultrasound diagnosis, ultrasound measurement, tissue ablation, or the like.

In this way, the wafer scale fabrication enables simultaneous fabrication of an array of transducers 200 utilizing at least some common layers or steps, reduces manual processes, improves volume and throughout of fabrication, and reduces fabrication time and costs.

As used herein, any of the x-axis, the y-axis, and the z-axis may correspond, respectively, to an azimuth direction, an elevation direction, and a propagation (or axial) direction. That is, it should be understood that the axes are arbitrary and may correspond to any axes of the ultrasound transducer 104 depending on orientation of the axes and/or the transducer 104. As used herein, “contact” may refer to indirect contact between a first component and a second component via the inclusion of an intermediate third component, or may refer to direct contact between the first component and the second component without the inclusion of any intermediate third components. As used herein, “provided on” may refer to a first component contacting a second component.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A transducer of an ultrasound probe, the transducer comprising:
a piezoelectric layer configured to transmit an ultrasound signal towards a region of interest of a subject to be imaged, and receive an echo signal reflected by the region of interest of the subject to be imaged;
a first electrode provided on a first surface of the piezoelectric layer;
a second electrode provided on a second surface of the piezoelectric layer;
a circuit that is electrically connected to the first electrode and the second electrode; and
a frame that is provided on at least a third surface of the piezoelectric layer, and that electrically connects the first electrode to the circuit.

2. The transducer of claim 1, wherein the frame is provided on the third surface of the piezoelectric layer and a fourth surface of the piezoelectric layer.

3. The transducer of claim 1, wherein the frame is provided on the third surface of the piezoelectric layer, a fourth surface of the piezoelectric layer, a fifth surface of the piezoelectric layer, and a sixth surface of the piezoelectric layer.

4. The transducer of claim 1, wherein the transducer is a one-dimensional array of transducer elements.

5. The transducer of claim 1, further comprising:
one or more acoustic matching layers configured to reduce an acoustic impedance between the subject and the transducer.

6. The transducer of claim 1, further comprising:
an acoustic dematching layer configured to attenuate the ultrasound signal, wherein the second electrode is provided between the acoustic dematching layer and the piezoelectric layer.

7. The transducer of claim 1, further comprising:
a lens configured to direct the ultrasound signal towards the region of interest of the subject.

8. The transducer of claim 1, further comprising:
a backing layer configured to attenuate ultrasound signals directed from the piezoelectric layer in a direction opposite to the subject.

9. The transducer of claim 1, wherein the transducer is formed by:
providing a frame array, including the frame, on a carrier,
providing the piezoelectric layer in a slot of the frame array, and
singulating the transducer, including the frame, from the frame array.

10. The transducer of claim 9, wherein the carrier is an acoustic matching layer.

11. The transducer of claim 9, wherein the carrier is an acoustic dematching layer.

12. The transducer of claim 9, wherein the carrier is a sacrificial substrate that is not a part of the transducer.

13. The transducer of claim 1, wherein the transducer is formed by:
providing a frame array including a plurality of slots on a carrier,
providing a plurality of piezoelectric layers in respective slots of the plurality of slots to form an array of transducers, and
singulating the transducer from the array of transducers.

14. The transducer of claim 1, wherein the piezoelectric layer is formed of a single crystal piezoelectric material.

15. The transducer of claim 1, wherein sidewalls of the piezoelectric layer are not metallized.

16. The transducer of claim 1, wherein:
the frame is a portion of a frame array used during manufacturing of the transducer,
the frame array includes a plurality of slots corresponding to respective transducers, and
the piezoelectric layer is provided in a slot, of the plurality of slots, of the frame array.

17. The transducer of claim 1, wherein the frame is formed of an electrically conductive material.

18. The transducer of claim 1, wherein the frame is formed of graphite.

19. An ultrasound probe comprising a transducer, the transducer comprising:
a piezoelectric layer configured to transmit an ultrasound signal towards a region of interest of a subject to be imaged, and receive an echo signal reflected by the region of interest of the subject to be imaged;
a first electrode provided on a first surface of the piezoelectric layer;
a second electrode provided on a second surface of the piezoelectric layer;
a circuit that is electrically connected to the first electrode and the second electrode; and
a frame that is provided on at least a third surface of the piezoelectric layer, and that electrically connects the first electrode to the circuit.

20. A method of manufacturing a transducer of an ultrasound probe, the method comprising:
providing a frame array on a carrier, wherein the frame array includes a plurality of slots;
providing a plurality of piezoelectric layers in respective slots of the plurality of slots to form an array of transducers; and
singulating the transducer from the array of transducers, wherein each transducer of the array of transducers comprises:
a respective piezoelectric layer, of the plurality of piezoelectric layers, configured to transmit an ultrasound signal towards a region of interest of a subject to be imaged, and receive an echo signal reflected by the region of interest of the subject to be imaged;
a first electrode provided on a first surface of the piezoelectric layer;
a second electrode provided on a second surface of the piezoelectric layer;
a circuit that is electrically connected to the first electrode and the second electrode; and
a respective frame, of the frame array, that is provided on at least a third surface of the piezoelectric layer, and that electrically connects the first electrode to the circuit.
